(19) United States
(12) Patent Application Publication
(10) Pub. No.: US 2003/0186880 A1
(43) Pub. Date: Oct. 2, 2003

(54) Combination therapy using trefoil peptides

(76) Inventor: Daniel K. Podolsky, Wellesley, MA (US)

Correspondence Address: CLARK & ELBING LLP, 101 FEDERAL STREET, BOSTON, MA, 02110, US

(21) Appl. No.: 10/397,953

(22) Filed: Mar. 26, 2003

Related U.S. Application Data
(60) Provisional application No. 60/367,574, filed on Mar. 26, 2002.
(60) Provisional application No. 60/422,708, filed on Oct. 31, 2002.

(51) Int. Cl. A61K 38/17; A61K 31/7048; A61K 31/33; A61K 31/66; A61K 31/525

(52) U.S. Cl. 514/12; 514/27; 514/49; 514/110; 514/183; 514/251

(57) ABSTRACT

This invention features methods and compositions of combination therapy suitable for treating and preventing epithelial lesions. Lesions are treated or prevented by combining trefoil peptide therapy with another medically useful therapeutic agent. Suitable therapeutics for combination therapy with a trefoil peptide included chemotherapeutics, particularly orally administered chemotherapeutics, analgesic, antibiotic, and anti-inflammatory agents. Useful trefoil peptides include the naturally occurring trefoil peptides intestinal trefoil factor, spasmolytic polypeptide (SP), and pS2, as well as non-naturally occurring peptides that contain a trefoil domain.

This application claims benefit of the filing date of the co-pending U.S. Provisional Application Nos. 60/367,574 (filed Mar. 26, 2002) and 60/422,708 (filed Oct. 31, 2002), hereby incorporated by reference.

### BACKGROUND OF THE INVENTION

Cancer is a broad term, encompassing more than one hundred different disease states. Cancers are characterized by abnormal and/or uncontrolled cell growth which may be triggered by a host of factors including chemicals, radiation, viruses, and unidentified environmental factors. Additionally, the incidence and progression of a cancer is influenced by physiological and biological factors unique to each patient including genetic make-up, hormone levels, and nutritional and immune status.

Chemical antineoplastic agents (chemotherapeutics) prevent the development, maturation, and spread of cancerous cells. Chemotherapeutic regimens currently utilize a wide range of products include alkylating agents, antimetabolites, hormone agonists and antagonists, nitrosoureas, and plant alkaloids. Chemotherapy is, however, often limited by the nature and severity of adverse side effects. Thus, as the focus of anti-cancer therapy shifts from short-term curative therapy to long-term disease management, adjunct therapies which reduce or eliminate adverse effects are required.

### SUMMARY OF THE INVENTION

This invention features therapeutic methods and compositions in which epithelial lesions are reduced. The invention employs pharmaceutical compositions containing at a trefoil peptide and at least one other therapeutic agent.

Accordingly, the invention provides a pharmaceutical composition containing (i) a trefoil peptide, and (ii) at least one therapeutic agent. The compositions can be formulated for delivery by any appropriate route of administration (e.g., oral, parenteral, transdermal, ocular, or rectal); however, compositions suitable for oral or intravenous administration are most preferred.

The invention also features a method for reducing adverse effects resulting from a medical intervention that damages mucosal epithelial cells by administering a trefoil peptide within 14 days, 10 days, 7 days, 5 days, 3 days, 24 hours, 12 hours, 1 hour, or simultaneously with the medical intervention. Antineoplastic therapy (i.e., surgical tumor resection, chemotherapy, and radiation therapy), for example, is a medical intervention known to be particularly damaging to mucosal epithelial cells in which trefoil peptide therapy is indicated. When the antineoplastic therapy is chemotherapy, the chemotherapeutic may be administered in the same or different pharmaceutical compositions. When administered in different compositions, the compositions need not be administered by the same route, at the same time, or for the same duration.

Therapeutic methods and compositions of the invention can be combinations of any therapeutic agent with a trefoil peptide. Preferably, therapeutic agents are chemotherapeutic, antibacterial, antifungal, antiviral, analgesic, or anti-inflammatory agents. More preferably, the therapeutic agent is a chemotherapeutic agent, particularly a chemotherapeutic suitable for oral administration, and is selected from the group consisting of busulfan, temozolomide, etoposide, melphalan, 5-fluorouracil, capecitabine, cyclophosphamide, methotrexate, and imatinib mesylate. Most preferably, the chemotherapeutic agent is imatinib mesylate.

Preferred routes of therapeutic delivery are by oral administration (e.g., pill, capsule, tablet, or syrup) or intravenous injection; however, subcutaneous, intramuscular, ophthalmic, vaginal, rectal, or topical administration are appropriate when clinically indicated.

Adverse effects caused by medical interventions (e.g., chemotherapy) which are effectively treated or prevented include any epithelial lesion including lesions of the alimentary canal epithelium and the vascular epithelium. Specifically, epithelial lesions particularly amenable to treatment using the methods of this invention include, for example, mucositis, enteritis, colitis, mucosal irritancy, and phlebitis.

The methods and compositions of the present invention utilize any therapeutic agent at doses known to be clinically effective. However, because adverse effects frequently limit the maximum tolerated of many therapeutics, particularly chemotherapeutics, combination therapy using a trefoil peptide may permit the administration of higher therapeutic doses than are possible in the absence of trefoil peptide therapy.

By “trefoil peptide” (TP) is meant any polypeptide having at least a trefoil domain (TD) and retaining a biological activity characteristic of the naturally occurring trefoil peptides. Thus, preferred TPs may be any mammalian homolog or artificial polypeptide that are substantially identical to human spasmolytic polypeptide (hSP; also known as TFF2, GenBank Accession No. NM_{—}005423; SEQ ID NO:5), human pS2 (also known as TFF1, GenBank Accession No. XM_{—}009779; SEQ ID NO:3), human intestinal trefoil factor (hITF; also known as TFF3, SEQ ID NO:1), and biologically active fragments thereof. If desired, the TP may contain a cysteine residue outside of the trefoil domain suitable for disulfide bonding in the formation of homo- and heterodimers. Most preferably, the additional cysteine is C-terminal to the trefoil domain. Exemplary TPs include ITF₁₅₋₇₃, ITF₁₋₆₂, ITF₁₋₇₀, ITF₁₋₇₂, ITF₂₅₋₇₃, ITF₁₋₇₃ and ITF₂₁₋₇₃. Preferably, a TP is encoded by a nucleic acid molecule that hybridizes under high stringency conditions to the coding sequence of hITF (SEQ ID NO:2), hSP (SEQ ID NO:6), or hpS2 (SEQ ID NO:4). TPs amenable to methods of this invention may exist as monomers, dimers, or multimers. For example, TP monomers may form an interchain disulfide linkage to form a dimer.

By “trefoil domain” is meant a polypeptide having a sequence substantially identical to any one of SEQ ID NOs:7-10, which correspond to the trefoil domains of hpS2₃₀₋₇₀, hSP₃₀₋₇₁, hSP₈₀₋₁₂₀, and hITF₂₄₋₆₄, respectively, and retain at least one biologic activity characteristic of trefoil peptides. The aligned polypeptide sequences of the four identified human trefoil domains are shown in FIG. 4. It is recognized in the art that one function of the six conserved cysteine residues is to impart the characteristic three-loop (trefoil) structure to the protein. The loop structure conforms to the general intrachain disulfide configuration of cys₁-cys₅ (corresponding to amino acid residues 25 and 51 of hITF; SEQ ID NO:1), cys₂-cys₄ (corresponding to amino acid residues 35 and 50 of hITF; SEQ ID NO:1), and cys₃-cys₆ (corresponding to amino acid residues 45 and 62 of hITF; SEQ ID NO:1).

By “chemotherapeutic” is meant any chemical which is administered to a patient, preferably a human patient, to provide antineoplastic therapy. Particularly useful classes of chemotherapeutics include alkylating agents, antimetabolites, hormone agonists and antagonists, nitrosoureas, and plant alkaloids. Most preferable are chemotherapeutics which are effective when administered orally such as busulfan, temozolomide, etoposide, melphalan, 5-fluorouracil, capecitabine, cyclophosphamide, methotrexate, and imatinib mesylate.

By “adverse effect” is meant any complication, undesired biological activity, or collateral toxicity associated with a medical procedure or therapy (e.g., antineoplastic chemotherapy). The adverse effects most influenced by the methods and compositions of the present invention are those which are caused by disruption of epithelial cells, including but not limited to gastrointestinal, skin, ocular, urogenital, respiratory, and cardiovascular epithelial cells. Typical adverse effects which may be mitigated or eliminated by the present invention include, for example, dermal reactions including photosensitivity, rash, radiation recall, erythema palmar-plantar erythrodysesthesia (skin eruptions characterized by swelling, pain, and erythema). Gastrointestinal adverse effects include mucositis, stomatitis, colitis, hemorrhagic enteritis, intestinal perforation, constipation, diarrhea, nausea, and dyspepsia. Other adverse effects that may be alleviated include pulmonary edema, chronic obstructive pulmonary disease (COPD), phlebitis, and conjunctivitis.

By “co-formulated” is meant any single pharmaceutically acceptable composition which contains two or more therapeutic or biologically active agents, such as a trefoil peptide and a chemotherapeutic. The most common co-formulations are compositions suitable for oral administration, such as solutions, suspensions, pills, capsules, or tablets, where each unit contains a plurality of therapeutic agents.

By “pharmaceutical preparation” or “pharmaceutically acceptable composition” is meant any composition suitable for administration to a patient, by any route, where the composition contains at least one therapeutically or biologically active agent and a pharmaceutically acceptable excipient. Typical pharmaceutical preparations include, but are not limited to pills, capsules, tablets, and syrups for oral administration, and buffered aqueous solutions for intravenous, intramuscular, or subcutaneous injection. An oral viscous solution, in the form of a spray or atomized mist may be used to administer to the buccal cavity. Suppositories can be used for intravaginal or rectal administration. Nebulizer solutions and multidose inhaler preparations are suitable for inhalation administration and ophthalmic drops are appropriate for ocular delivery. Any of these formulations can be prepared by well known and accepted methods of art. See, for example, Remingtion: The Science and Practice of Pharmacy, 19^{th} edition, (ed. A R Gennaro), Mack Publishing Co., Easton, Pa., 1995.

By “biologically active,” when referring to a TP is meant any polypeptide that exhibits an activity common to naturally occurring trefoil peptides. An example of a biological activity common to the family of trefoil peptides is the ability to alter gastrointestinal motility in a mammal. Other biological activities include mucopolysaccaride binding, maintenance of the mucosa, and repair of mucosal integrity upon injury (see, for example, Taupin et al., Proc. Natl. Acad. Sci, USA, 97:799-804, 1999).

By “substantially identical”, when referring to a trefoil domain of SEQ ID NOs.:7-10 is meant an amino acid sequence that has 85%, 90%, 95%, or 99% sequence identity to the sequence of a reference amino acid. It is recognized in the art that a polypeptide may also be substantially identical if an amino acid sequence differs only by conservative amino acid substitutions, for example, substitution of one amino acid for another of the same class (e.g., any of the hydrophobic amino acids can be substituted for each other, i.e., methionine, valine, alanine, isoleucine and leucine; arginine for lysine, etc.). For polypeptides, the length of comparison sequences will generally be at least 30 amino acids, preferably at least 40 amino acids, more preferably at least 50 amino acids, and most preferably at least 60 amino acids. For nucleic acids, the length of comparison sequences will generally be at least 60 nucleotides, preferably at least 90 nucleotides, and more preferably at least 120 nucleotides.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65° C., or a buffer containing 48% formamide, 4.8× SSC, 0.2 M Tris-Cl, pH 7.6, 1× Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well known by those skilled in the art of molecular biology. See, e.g., F. Ausubel et al., in Current Protocols in Molecular Biology, John Wiley & Sons, New York, N.Y., 1998, hereby incorporated by reference.

By “isolated DNA” is meant DNA that is free of the genes which, in the naturally-occurring genome of the organism from which the given DNA is derived, flank the DNA. Thus, the term “isolated DNA” encompasses, for example, cDNA, cloned genomic DNA, and synthetic DNA.

Other features and advantages of the invention will be apparent from the following detailed description, and from the claims.

### BRIEF DESCRIPTION OF THE DRAWINGS

FIGS. 1A-B show the amino acid sequence (Accession No. BAA95531; SEQ ID NO.:1) and cDNA sequence (GenBank Accession No. NM_{—}003226; SEQ ID NO.:2) of human intestinal trefoil factor (hITF), respectively.

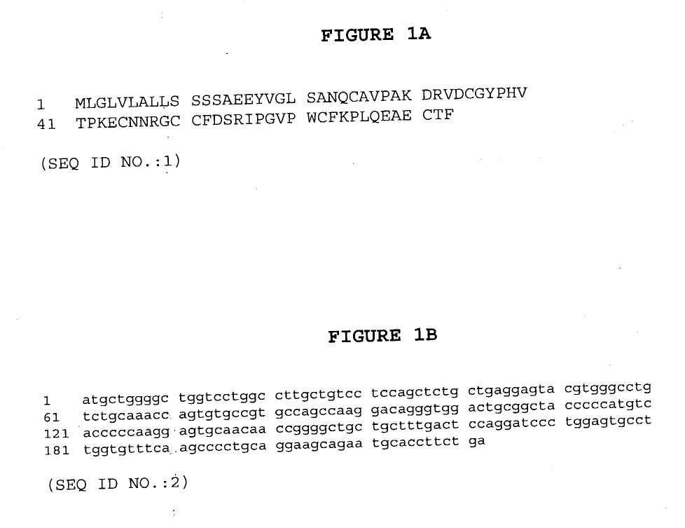

FIGS. 2A and 2B show the amino acid sequence (Accession No. NP_{—}0032166, SEQ ID NO.:3) and cDNA sequence (SEQ ID NO.:4) of human pS2 (hpS2) protein, respectively.

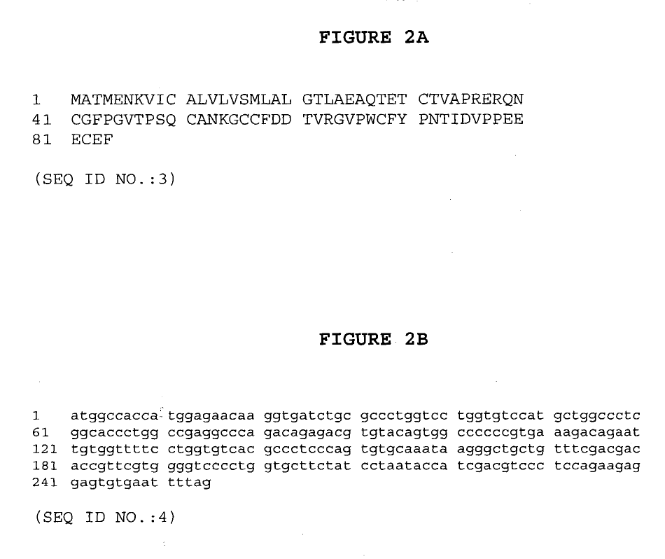

FIGS. 3A and 3B show the amino acid sequence (Accession No. 1909187A; SEQ ID NO.:5) and cDNA sequence (SEQ ID NO.: 6) of human spasmolytic polypeptide (hSP).

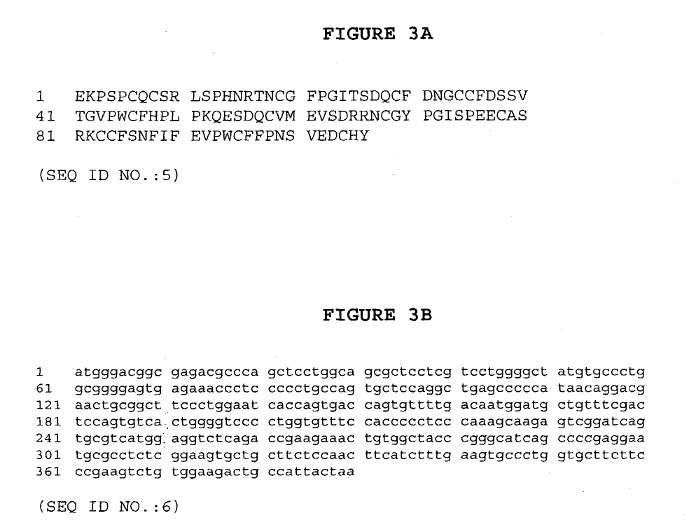

FIG. 4 is a multisequence alignment of trefoil domains (SEQ ID NOS.:7-10) hsP2, hSP, and hITF. X denotes any amino acid residue.

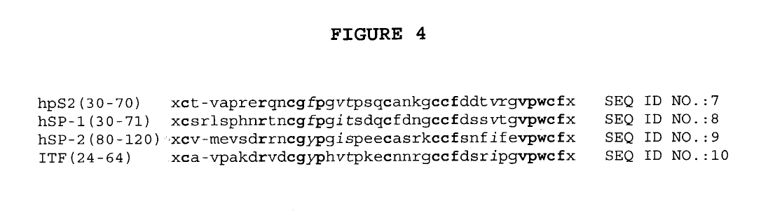

### DETAILED DESCRIPTION

Many antineoplastic chemotherapies are designed to destroy rapidly dividing cancer cells. Adverse effects from chemotherapy are often dose limiting and frequently occur as a consequence of collateral damage to other proliferative but non-cancerous cell populations in the body. Particularly susceptible proliferative cell populations include the epithelial cells of the gastrointestinal, respiratory, and urinary tracts, and the dermis and epidermis.

Mucositis is a common adverse effect associated with chemotherapy which is characterized by inflammation of the mucous membranes, particularly in the oral cavity and gastrointestinal (GI) tract. The intestinal crypt cells are highly mitotically active and most susceptible to disruption by chemotherapy. Symptoms of mucositis include ulcerations, redness, and swelling leading to dehydration and malnutrition, pain, nausea, vomiting, abdominal cramping, and diarrhea. In severe cases, mucositis can be so debilitating that patients may require prolonged hospitalization, parenteral nutrition, and narcotic pain medication. Additionally, destruction of the GI mucous membrane increases a patient's susceptibility to local and systemic infection and sepsis. Disruption of the barrier function permits entry of microorganisms and microbial products normally retained in the gut lumen. Thus, pharmaceutical preparations which reduce the adverse effects associated with chemotherapy will improve the patient's quality of life, compliance with self-medication, and may permit administration of higher chemotherapeutic doses.

Mammalian trefoil peptides were discovered in 1982. One of the mammalian trefoil peptides, human intestinal trefoil factor (ITF) has been characterized extensively, and is described in U.S. Pat. Nos. 6,063,755, and 6,221,840, hereby incorporated by reference. The other two known human trefoil peptides are spasmolytic polypeptide (SP) and pS2. Trefoil peptides, described extensively in the literature (e.g., Sands et al., Annu. Rev. Physiol. 58: 253-273 (1996), hereby incorporated by reference), are expressed in the gastrointestinal tract and have a three-loop structure formed by intrachain disulfide bonds between conserved cysteine residues. These peptides protect the intestinal tract from injury and can be used to treat intestinal tract disorders such as peptic ulcers and inflammatory bowel disease. Homologs of these human peptides have been found in a number of non-human animal species. All members of this protein family, both human and non-human, are referred to herein as trefoil peptides. Human ITF will be referred to most extensively in this application; however, the activity of human ITF is common to each of the mammalian trefoil peptides.

Production of Trefoil Peptides

Trefoil peptides can be produced by any method known in the art for expression of recombinant proteins. For example, the isolated nucleic acids that encode trefoil peptides or fragments thereof can be cloned into a mammalian expression vector. Appropriate vectors include pMAMneo (Clontech, Palo Alto, Calif.) which provides a RSV-LTR enhancer linked to a dexamethasone-inducible MMTV-LTR promoter, an SV40 origin of replication (allows replication in COS cells), a neomycin gene and SV40 splicing and polyadenylation sites. This vector can be used to express the protein in COS cells, CHO cells, or mouse fibroblasts. The gene may also be cloned into a vector for expression in Drosophila cells using the baculovirus expression system. These methods of production are illustrative of techniques known in the art, and are not intended to be limiting.

Formulations

The invention features combination therapy using a trefoil peptide and an antineoplastic chemotherapeutic. Optionally, other therapeutics such as antibiotics (antibacterial, antifungal, or antiviral agents), anti-inflammatories, or analgesics, may be included. The preferable route of administration for all therapeutics is per os; however, one or more of the therapeutics can be administered by other routes including, for example, parenteral injection (i.e. intravenous, intramuscular, or subcutaneous), rectal or vaginal suppository, topical application, or ophthalmic preparation. For ease of delivery and the maximization of patient compliance when self-medication is required, preferably, a single formulation containing every active therapeutic is administered. Alternatively, the therapeutics can be administered in separate formulations using the same or different routes of administration, allowing precise control over the timing and dosage of each therapeutic component. For example, the chemotherapeutic is delivered by intravenous injection once every two weeks and a capsule containing the trefoil peptide is ingested daily.

Oral Administration

The most preferred formulation is, therefore, a single formulation, suitable for ingestion, which contains both a chemotherapeutic and a trefoil peptide. The formulation can be, for example, a pill, capsule, tablet, emulsion, solution, suspension, syrup, or soft gelatin capsule. Methods well known in the art for making formulations are found, for example, in Remington's Pharmaceutical Sciences (19th edition), ed. A. Gennaro, 1995, Mack Publishing Company, Easton, Pa.

One skilled in the art will recognize that the trefoil peptide and chemotherapeutic can be administered orally in a sustained release composition, such as those described in, for example, U.S. Pat. No. 5,672,659 and U.S. Pat. No. 5,595,760. The use of immediate or sustained release compositions depends on the type of condition being treated, the adverse effect being treated or prevented, and the pharmacokinetic characteristics of the therapeutics.

In other embodiments, formulations which target the therapeutic release to particular regions of the gastrointestinal tract can be prepared. For example, the therapeutics can be formulated in cellulosic rafts or other delivery vehicles having sustained release characteristics and are retained in the stomach for extended periods of time (see, for example, U.S. Pat. Nos. 4,946,685 and 6,261,601). Alternatively, the chemotherapeutic and the trefoil peptide can be encapsulated in an enteric coating which prevents release degradation and release from occurring in the stomach, but dissolves readily in the mildly acidic or neutral pH environment of the small intestine. A formulation targeted for release of drug to the colon, utilizing technologies such as time-dependent or pH enzymatic erosion of polymer matrix or coating, osmotic pumps, magnetic or radio frequency-induced release can also be used.

For particular therapies, the chemotherapeutic and trefoil peptide may be released in different regions of the gastrointestinal tract. A multilayer formulation having different release characteristics between the layers can be prepared. For example, an inner core, containing the chemotherapeutic is prepared and encapsulated in an enteric coating. An outer layer containing the trefoil peptide is then added. This formulation has the advantage of releasing the acid-stable trefoil peptide in the stomach to provide enhanced treatment of gastric mucosal lesions while preserving the integrity of the inner core for chemotherapeutic release in the small or large intestine. The trefoil peptide may be complexed, by inclusion, ionic association, hydrogen bonding, hydrophobic bonding, or covalent bonding with another species in order to modify its targeted delivery properties. In addition polymers or complexes susceptible to enzymatic or microbial lysis may also be used as a means to deliver drug. Alternatively, the two-stage release formulation may consist of acid stable microspheres, encapsulating the therapeutic to be released later in the lower gastrointestinal tract admixed with an immediate release formulation of the other therapeutic. Microspheres can be made by any appropriate method, or from any pharmaceutically acceptable material. Particularly useful are proteinoid microspheres (see, for example, U.S. Pat. Nos. 5,601,846, or 5,792,451) and PLGA-containing microspheres (see, for example, U.S. Pat. No. 6,235,224).

Multilayer or other pharmaceutical formulations, such as those described above, which physically separate the trefoil peptide from the other therapeutics can be used to prevent chemical reactions between the therapeutics. For example, trefoil peptides form a characteristic three loop structure as a result of intramolecular disulfide bonding between conserved cysteine residues. It is, therefore, desirable to maintain the sulfur-containing amino acids in a reduced state. Thus, when co-formulations require a trefoil peptide and an oxidizing agent, it is preferable to keep the two therapeutics physically separated in order to maintain the potency of each.

Parenteral Administration

In another particularly desirable embodiment, the chemotherapeutic and the trefoil peptide are co-formulated in a single preparation suitable for parenteral delivery such as intravenous injection. In addition to the increased convenience, the presence of the trefoil peptide in the chemotherapeutic preparation reduces the incidence and severity of phlebitis at the site of chemotherapeutic injection. Alternatively, for intravenous administration of a chemotherapeutic, a small amount of the trefoil peptide is the formulation in order to prevent phlebitis at the infusion site, but high dose trefoil peptide therapy is administered orally.

Dosages

The trefoil peptide used in the methods and compositions of the invention should be provided in therapeutically effective amounts. Preferably, patients will be administered 1, 10, 50, 100, 250, or 500 mg of trefoil peptide once, twice, or three times each day. Trefoil peptide therapy will continue until the epithelial lesion is healed, or for the duration of chemotherapy. Typically, the duration of therapy will be for one week to one month; however, therapy may be required for as much as one year, or even the lifetime of the patient.

Chemotherapeutic agents employed in the pharmaceutical preparations and therapies of the present invention can be used in the dose ranges currently known and used for those agents. However, because the adverse effects treated using the trefoil peptide frequently limit the maximum tolerated dose of chemotherapeutic, the combination therapy of this invention may allow the chemotherapeutic to be dosed at a higher level than would otherwise be used. Trefoil peptide and chemotherapeutic dosages may be altered depending on the clinical condition of the patient, the type of cancer, and anticipated severity of the adverse effects. Additional considerations in dose selection include: disease etiology, patient age (pediatric, adult, geriatric), general health and comorbidity. Table 1 provides exemplary chemotherapeutics known to be effective when administered orally; however, any effective chemotherapeutic is can be combined with the trefoil peptides according to the methods of this invention, regardless of the route of administration.

TABLE 1
Exemplary Oral Chemotherapeutics

| Chemotherapeutic | Oral Dosages | |
|---|---|---|
| Busulfan | 1.0-12.0 | mg/m²/day |
| Temozolomide | 100-200 | mg/m²/day |
| Etoposide | 50-200 | mg/m²/day |
| Melphalan | 4.0-12.0 | mg/m²/day |
| Capecitabine | 625-2500 | mg/m²/day |
| Cyclophosphamide | 1.0-5.0 | mg/kg/day |
| Methotrexate | 0.625-2.5 | mg/kg/day |
| Imatinib mesylate | 400-600 | mg/day |

Other Embodiments

It is recognized by persons of skill in the art that, when used in conjunction with antineoplastic chemotherapy, the methods and compositions described herein can, optionally, include one or more additional therapeutics, as clinically indicated. Other useful therapeutics that can be used with the methods and compositions of this invention include, for example, antibiotics including antibacterials, antivirals, and antifungal, anti-inflammatories, and analgesics. Additional therapeutics can be administered in separate formulations or can be co-formulation with any one or more of the therapeutic compositions described herein.

Further, formulations suitable for orally administration which contain a trefoil peptide and a therapeutics other than chemotherapeutic are useful for treating lesions of the gastrointestinal tract and other conditions in patients not receiving antineoplastic therapy.

What is claimed is:

1. A pharmaceutical composition comprising (i) a trefoil peptide, and (ii) a therapeutic agent.

2. The composition of claim 1, wherein said therapeutic agent is a chemotherapeutic agent.

3. The composition of claim 2, wherein said chemotherapeutic agent is selected from the group consisting of busulfan, temozolomide, etoposide, melphalan, 5-fluorouracil, capecitabine, cyclophosphamide, methotrexate, and imatinib mesylate.

4. The composition of claim 3, wherein said chemotherapeutic is imatinib mesylate.

5. The composition of claim 1, wherein said composition is suitable for oral or intravenous administration.

6. The composition of claim 1, wherein said trefoil peptide is spasmolytic polypeptide, pS2, intestinal trefoil factor, or a biologically active fragment thereof.

7. The composition of claim 6, wherein said trefoil peptide is ITF₁₋₇₃, ITF₁₅₋₇₃, or ITF₂₁₋₇₃.

8. The composition of claim 1, wherein said trefoil peptide comprises a polypeptide having a sequence substantially identical to SEQ ID NOs: 7-10.

9. The composition of claim 1, wherein said therapeutic agent is an anti-inflammatory agent.

10. The composition of claim 1, wherein said therapeutic agent is an antibacterial, antiviral, or antifungal agent.

11. The composition of claim 1, wherein said therapeutic agent is an analgesic.

12. A method for reducing an adverse effect in a patient to whom a chemotherapeutic agent is administered, said method comprising administering to said patient a trefoil peptide within fourteen days of said chemotherapeutic agent in an amount sufficient to reduce said adverse effect.

13. The method of claim 12, wherein said trefoil peptide and said chemotherapeutic agent are administered simultaneously.

14. The method of claim 13, wherein said trefoil peptide and said chemotherapeutic agent are present in one pharmaceutically acceptable composition.

15. The method of claim 14, wherein said composition is suitable for oral or intravenous administration.

16. The method of claim 12, wherein said trefoil peptide is spasmolytic polypeptide, pS2, intestinal trefoil factor, or a biologically active fragment thereof.

17. The method of claim 12, wherein said trefoil peptide is ITF₁₋₇₃, ITF₁₅₋₇₃, or ITF₂₁₋₇₃.

18. The method of claim 12, wherein said trefoil peptide comprises a polypeptide having a sequence substantially identical to SEQ ID NOs: 7-10.

19. The method of claim 12, wherein said adverse effect is mucositis.

20. The method of claim 12, wherein said adverse effect is enteritis or colitis.

21. The method of claim 12, wherein said adverse effect is mucosal irritancy.

22. The method of claim 12, wherein said adverse effect is phlebitis.

23. The method of claim 12, wherein said trefoil peptide and said chemotherapeutic agent are administered in different pharmaceutically acceptable compositions.

24. The method of claim 23, wherein said trefoil peptide is administered prior to said chemotherapeutic agent.

25. The method of claim 23, wherein said trefoil peptide is administered subsequent to said chemotherapeutic agent.


SEQUENCE LISTING (10 sequences)

SEQ ID NO: 1 - protein, 73 aa, Homo sapiens
MLGLVLALLSSSSAEEYVGLSANQCAVPAKDRVDCGYPHVTPKECNNRGCCFDSRIPGVPWCFKPLQEAECTF

SEQ ID NO: 2 - DNA, 222 bp, Homo sapiens
atgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctga

SEQ ID NO: 3 - protein, 84 aa, Homo sapiens
MATMENKVICALVLVSMLALGTLAEAQTETCTVAPRERQNCGFPGVTPSQCANKGCCFDDTVRGVPWCFYPNTIDVPPEEECEF

SEQ ID NO: 4 - DNA, 255 bp, Homo sapiens
atggccaccatggagaacaaggtgatctgcgccctggtcctggtgtccatgctggccctcggcaccctggccgaggcccagacagagacgtgtacagtggccccccgtgaaagacagaattgtggttttcctggtgtcacgccctcccagtgtgcaaataagggctgctgtttcgacgacaccgttcgtggggtcccctggtgcttctatcctaataccatcgacgtccctccagaagaggagtgtgaattttag

SEQ ID NO: 5 - protein, 106 aa, Homo sapiens [sequence omitted]

SEQ ID NO: 6 - DNA, 390 bp, Homo sapiens
atgggacggcgagacgcccagctcctggcagcgctcctcgtcctggggctatgtgccctggcggggagtgagaaaccctccccctgccagtgctccaggctgagcccccataacaggacgaactgcggcttccctggaatcaccagtgaccagtgttttgacaatggatgctgtttcgactccagtgtcactggggtcccctggtgtttccaccccctcccaaagcaagagtcggatcagtgcgtcatggaggtctcagaccgaagaaactgtggctacccgggcatcagccccgaggaatgcgcctctcggaagtgctgcttctccaacttcatctttgaagtgccctggtgcttcttcccgaagtctgtggaagactgccattactaa

SEQ ID NO: 7 - protein, 41 aa, Artificial Sequence
  Synthetic
XCTVAPRERQNCGFPGVTPSQCANKGCCFDDTVRGVPWCFX

SEQ ID NO: 8 - protein, 42 aa, Artificial Sequence
  Synthetic
XCSRLSPHNRTNCGFPGITSDQCFDNGCCFDSSVTGVPWCFX

SEQ ID NO: 9 - protein, 41 aa, Artificial Sequence
  Synthetic
XCVMEVSDRRNCGYPGISPEECASRKCCFSNFIFEVPWCFX

SEQ ID NO: 10 - protein, 41 aa, Artificial Sequence
  Synthetic
XCAVPAKDRVDCGYPHVTPKECNNRGCCFDSRIPGVPWCFX